(19) United States
(12) Patent Application Publication
(10) Pub. No.: US 2024/0261053 A1
(43) Pub. Date: Aug. 8, 2024

(54) WEARABLE TUBE HOLDING DEVICE

(71) Applicant: Pamela Lightburn, Acworth, GA (US)

(72) Inventor: Pamela Lightburn, Acworth, GA (US)

(21) Appl. No.: 18/106,100

(22) Filed: Feb. 6, 2023

(51) Int. Cl. A61B 50/20; A45F 5/00

(52) U.S. Cl. CPC A61B 50/20; A45F 5/00; A45F 2005/008

(57) ABSTRACT

A wearable tube holding device, including a main body to be worn around a wrist of a user, and a plurality of elastic loops disposed on at least a portion of the main body to receive at least one tube therein.

### BACKGROUND

### 1. Field

The present general inventive concept relates generally to medical devices, and particularly, to a wearable tube holding device.

### 2. Description of the Related Art

In the medical field, blood drawing can be a nerve wrecking and anxiety inducing process for many patients, regardless of age. A phlebotomist's confidence and disposition, along with the preparation and efficiency of movement while drawing blood are essential aspects of comforting the patient. When the phlebotomist is constantly reaching for supplies when a needle is inserted within a patient can leave the patient even more anxious and can potentially lead to injury. Moreover, not having extra tubes within the immediate proximity disrupts the blood drawing process, which can be distracting to the phlebotomist.

Generally, the tubes used to connect to the needle are stored in a carton and/or a tray. However, the tray is inconvenient because it is often cumbersome to use and kept on a flat surface nearby.

Therefore, there is a need for a wearable tube holding device that stores the tubes and provides immediate access to the tubes during blood drawing.

### SUMMARY

The present general inventive concept provides a wearable tube holding device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a wearable tube holding device, including a main body to be worn around a wrist of a user, and a plurality of elastic loops disposed on at least a portion of the main body to receive at least one tube therein.

The main body may include an outer surface circumferentially disposed in a circle on at least a portion of a first side of the main body, an inner surface circumferentially disposed in a circle on at least a portion of a second side of the main body opposite with respect to the first side, and a wrist receiving aperture disposed within the main body to receive the wrist therethrough.

The outer surface may be more rigid under each of the plurality of elastic loops with respect to each portion of the outer surface outside a boundary of each of the plurality of elastic loops.

The wearable tube holding device may further include a friction assembly disposed on at least a portion of an inner surface of each of the plurality of elastic loops to prevent the at least one tube from moving within each of the plurality of elastic loops.

The friction assembly may be a foam surface to compress against a surface area of the at least one tube.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a side perspective view of a wearable tube holding device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the FIGURES, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Wearable Tube Holding Device 100
Main Body 110
Outer Surface 111
Inner Surface 112
Wrist Receiving Aperture 113
Elastic Loops 120
Friction Assembly 130

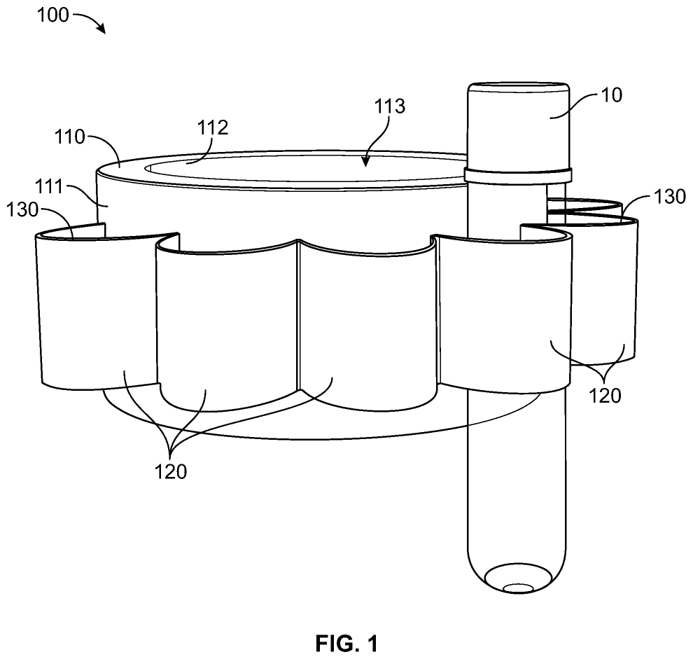

FIG. 1 illustrates a side perspective view of a wearable tube holding device 100, according to an exemplary embodiment of the present general inventive concept.

The wearable tube holding device 100 may be constructed from at least one of cloth, metal, plastic, and rubber, etc., but is not limited thereto.

The wearable tube holding device 100 may include a main body 110, a plurality of elastic loops 120, and a friction assembly 130, but is not limited thereto.

Referring to FIG. 1, the main body 110 is illustrated to have a cylindrical shape. However, the main body 110 may be rectangular, circular, conical, triangular, pentagonal, hexagonal, heptagonal, octagonal, or any other shape known to one of ordinary skill in the art, but is not limited thereto.

Referring again to FIG. 1, the main body 110 is illustrated to be a wristband. However, the main body 110 may be a bracelet, a ring, a necklace, a choker, an anklet, and/or a belt, but is not limited thereto. The main body 110 may have any size, such as 3.15 inches by 3.15 inches.

The main body 110 may include an outer surface 111, an inner surface 112, and a wrist receiving aperture 113, but is not limited thereto.

The outer surface 111 may have a smooth texture and/or be flexible. Additionally, the outer surface 111 may be circumferentially disposed in a circle on at least a portion of a first side of the main body 110.

The inner surface 112 may be circumferentially disposed in a circle on at least a portion of a second side of the main body 110 opposite with respect to the first side. Moreover, the inner surface 112 may have a diameter and/or a circumference less than a diameter and/or a circumference of the outer surface 111.

The main body 110 may be at least partially elastic, such that the main body 110 may at least partially deform (i.e., bend, stretch, expand) in response to inserting a wrist and/or a hand therethrough. In other words, the main body 110 may be worn around the wrist and/or the hand of a user.

The plurality of elastic loops 120 may be circumferentially disposed in a circle on at least a portion of the outer surface 112. Each of the plurality of elastic loops 120 may at least partially extend a distance away from the outer surface 112 in a convex shaped loop. Each of the plurality of elastic loops 120 may at least partially deform (i.e., bend, stretch, expand) in response to inserting at least one tube 10 therein. As such, each of the plurality of elastic loops 120 may receive and/or store the at least one tube 10 therein. Accordingly, the plurality of elastic loops 120 may facilitate use of the at least one tube 10, such that a phlebotomist may retrieve the at least one tube 10 from at least one of the plurality of elastic loops 120. The plurality of elastic loops 120 may have any size, such as 2 inches.

Optionally, the outer surface 112 may be more rigid under each of the plurality of elastic loops 120 with respect to each portion of the outer surface 112 outside a boundary of each of the plurality of elastic loops 120, such that the outer surface 112 may stabilize a position of the at least one tube 10.

The friction assembly 130 may include a foam surface, a plurality of bubbled protrusions extending away therefrom, an adhesive (e.g., a tape, glue), and/or any combination thereof, but is not limited thereto.

The friction assembly 130 may be disposed on at least a portion of an inner surface of each of the plurality of elastic loops 120. The friction assembly 130 may prevent the at least one tube 10 from moving within each of the plurality of elastic loops 120 absent an external application of force (e.g., pushing and/or pulling by the user). As such, the friction assembly 130 may prevent the at least one tube 10 from being lost while stored within each of the plurality of elastic loops 120. For example, the foam surface may compress and/or expand against a surface area of the at least one tube 10, the plurality of bubbled protrusions may increase friction due to each bubble connecting to the at least one tube 10, and/or the adhesive may affix and/or adhere to the surface area of the at least one tube 10.

Therefore, the wearable tube holding device 100 may store the at least one tube 10 within easy reach of the user. Also, the wearable tube holding device 100 may simplify blood drawing by allowing empty tubes to be retrieved, connected to a needle during a blood draw, and subsequently stored on the wearable tube holding device 100. Thus, the wearable tube holding device 100 may reduce a time needed for the phlebotomist to draw blood.

The present general inventive concept may include a wearable tube holding device 100, including a main body 110 to be worn around a wrist of a user, and a plurality of elastic loops 120 disposed on at least a portion of the main body 110 to receive at least one tube 10 therein.

The main body 110 may include an outer surface 111 circumferentially disposed in a circle on at least a portion of a first side of the main body 110, an inner surface 112 circumferentially disposed in a circle on at least a portion of a second side of the main body 110 opposite with respect to the first side, and a wrist receiving aperture 113 disposed within the main body 110 to receive the wrist therethrough.

The outer surface 111 may be more rigid under each of the plurality of elastic loops 120 with respect to each portion of the outer surface 111 outside a boundary of each of the plurality of elastic loops 120.

The wearable tube holding device 100 may further include a friction assembly 130 disposed on at least a portion of an inner surface of each of the plurality of elastic loops 120 to prevent the at least one tube 10 from moving within each of the plurality of elastic loops 120.

The friction assembly 130 may be a foam surface to compress against a surface area of the at least one tube 10.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A wearable tube holding device, comprising:
a main body to be worn around a wrist of a user; and
a plurality of elastic loops disposed on at least a portion of the main body to receive at least one tube therein.

2. The wearable tube holding device of claim 1, wherein the main body comprises:
an outer surface circumferentially disposed in a circle on at least a portion of a first side of the main body;
an inner surface circumferentially disposed in a circle on at least a portion of a second side of the main body opposite with respect to the first side; and
a wrist receiving aperture disposed within the main body to receive the wrist therethrough.

3. The wearable tube holding device of claim 2, wherein the outer surface is more rigid under each of the plurality of elastic loops with respect to each portion of the outer surface outside a boundary of each of the plurality of elastic loops.

4. The wearable tube holding device of claim 1, further comprising:
a friction assembly disposed on at least a portion of an inner surface of each of the plurality of elastic loops to prevent the at least one tube from moving within each of the plurality of elastic loops.

5. The wearable tube holding device of claim 4, wherein the friction assembly is a foam surface to compress against a surface area of the at least one tube.
